(19) United States
(12) Patent Application Publication
(10) Pub. No.: US 2026/0085022 A1
(43) Pub. Date: Mar. 26, 2026

(54) METHODS FOR CRACKING POLYOLEFINS USING UNSUPPORTED ACIDIC POLYOXOMETALATES

(71) Applicant: University of Kansas, Lawrence, KS (US)

(72) Inventors: Bala Subramaniam, Lawrence, KS (US); Hongda Zhu, Lawrence, KS (US)

(73) Assignee: University of Kansas, Lawrence, KS (US)

(21) Appl. No.: 19/196,923

(22) Filed: May 2, 2025

Related U.S. Application Data
(60) Provisional application No. 63/642,991, filed on May 6, 2024.

(51) Int. Cl. C07C 4/06; C10G 11/04; C10G 11/10; C10G 11/20

(52) U.S. Cl. CPC C07C 4/06; C10G 11/04; C10G 11/10; C10G 11/20; C07C 2523/30; C07C 2527/18; C07C 2527/188; C10G 2300/1003; C10G 2300/30

(57) ABSTRACT

Methods for cracking a polyolefin are provided which comprise heating a blend comprising a polyolefin and an unsupported acidic polyoxometalate in a reaction zone of a reactor chamber, the reaction zone at a reaction temperature, while flowing a gas through the blend, to induce carbon-carbon bond cleavage in the polyolefin and form a processed blend comprising cracked hydrocarbons; passing at least a portion of the processed blend to a reflux zone of the reactor chamber, the reflux zone at a reflux temperature; and collecting the cracked hydrocarbons.

### CROSS-REFERENCE TO RELATED APPLICATIONS

The present application claims priority to U.S. Provisional Patent Application No. 63/642,991 that was filed May 6, 2024, the entire contents of which are hereby incorporated by reference.

### REFERENCE TO GOVERNMENT RIGHTS

This invention was made with government support under 2119754 awarded by the National Science Foundation. The government has certain rights in the invention.

### BACKGROUND

The production of polyolefins including polyethylene and polypropylene contributes to more than 50% of non-fiber synthetic plastics, at a scale of over 150 million tons per year globally. While most of these are single-use plastics and end up in landfill, incinerators, or oceans, they can be potentially recycled as feedstock. Various approaches have been evaluated, including pyrolysis, hydrocracking, hydrogenolysis, acid-cracking, etc. and combinations of these methods for deconstructing these polyolefins to products such as paraffins, olefins, aromatics, dihydrogen, and special forms of carbon. Various catalysts have been used in these processes. However, deposition of carbonaceous species (the species are often referred to as “coke”, and the process as “coking”) is usually inevitable during cracking, including the formation of olefinic intermediates that undergo Diels-Alder or similar reactions forming aromatic coke. These catalysts are regenerated by burning the coke in air.

### SUMMARY

Methods for cracking a polyolefin (e.g., high-density polyethylene, HDPE) are provided which involve combining a polyolefin and an unsupported acidic polyoxometalate in a reactor chamber under conditions (e.g., heating and flowing gas) to induce C—C bond cleavage in the polyolefin to form products (e.g., cracked hydrocarbons).

The present disclosure is illustrated by reference to an Example, below, in which the catalytic cracking of high-density polyethylene (HDPE) was demonstrated over unsupported acidic W-based polyoxometalates in an up-flow fixed-bed reactor using flowing nitrogen. The product distribution was shown to depend on the catalyst pre-reduction method and conditions, the reactor temperature and the temperature in the reactor effluent reflux zone. For recycling the catalyst, it was dissolved in water to separate the spent catalyst from coke and no burning of the coke was required. Pre-reducing the catalyst was also found to mitigate coke formation by substrate (HDPE) dehydrogenation and also cased catalyst recovery. On a freshly H₂-prereduced catalyst, cracking activity at temperatures as low as 573 K was observed. At reactor and reflux temperatures of 623 K and 313 K respectively, ˜80% yield of predominantly paraffinic hydrocarbons with an average formula of C_{21.9}H_{45.9} was obtained. It was further discovered that Cr traces present in the HDPE substrate progressively accumulated in the recycled catalyst, leading to an increase in cracking activity. After five catalyst recycles, a Cr accumulation of ˜137 ppm in the catalyst shifts the product distribution to lower hydrocarbons with an average formula of C_{15.1}H_{31.9}. Higher Cr content was found to reduce the stability of the Keggin structure in the strongly reducing environment of H₂, and renders the catalyst insoluble in water and unrecoverable. However, in sharp contrast, catalyst pre-reduction by olefins instead of H₂ was able to preserve the structural integrity of W₁₂ clusters despite some extent of oxygen loss. Electron paramagnetic resonance (EPR) spectroscopy studies and computational results supported the hypothesis that both the incorporation of Cr, which is much harder than W and introduces an odd number of d electrons (similarly as olefin or PE reduction does), as well as the extent of reduction of the catalysts may affect the atomic and electronic structures of the heteropolytungstates, influencing their acidity and activity.

In embodiments, a method for cracking a polyolefin comprises: heating a blend comprising a polyolefin and an unsupported acidic polyoxometalate in a reaction zone of a reactor chamber, the reaction zone at a reaction temperature, while flowing a gas through the blend, to induce carbon-carbon bond cleavage in the polyolefin and form a processed blend comprising cracked hydrocarbons; passing at least a portion of the processed blend to a reflux zone of the reactor chamber, the reflux zone at a reflux temperature; and collecting the cracked hydrocarbons.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIG. 1. Product distributions from thermal pyrolysis (comparative) and catalytic pyrolysis according to an illustrative embodiment of the present methods using fresh and recovered catalysts. Selectivity values include mixed wax and liquid products. Conditions: carrier gas (N₂) flow: 300 std. cm³/min, HDPE loading: ˜10 g. For 6 h thermal pyrolysis: reflux zone temperature: ˜313 K. For 16 h catalytic pyrolysis: catalyst loading: ˜10 g catalyst, reflux zone temperature: ˜403 K.

FIG. 2. Product distributions from fresh and recovered catalyst pre-reduced by H₂. Note: Selectivity values are based on mixed wax and liquid products. Cr contents were determined by ICP-OES. Conditions: carrier gas (N₂) flow rate: 300 std. cm³/min, HDPE loading: ˜10 g, catalyst loading: ˜10 g, reaction temperature: 623 K, reflux zone temperature: ˜313 K, run time: 6 h.

FIG. 3, Spectra A-F. Powder XRD histograms of the samples produced from H₃PW₁₂O₄₀ hydrate following various histories. Note: The pre-reduction step and the catalytic runs were performed at 623 K. The two x axes are Bragg angle (θ) and interplanar spacing (d).

FIG. 4. Product distributions from fresh and recovered catalyst pre-reduced by HDPE. Note: Selectivity values are for mixed wax and liquid. Same conditions as in FIG. 2.

FIG. 5, Spectra A-F. Powder X-band EPR spectra of various catalyst samples collected at 7.4-7.9 K and respective simulated spectra. Spectrum A was collected with an empty cavity, and brief descriptions of the histories of the samples are labeled for spectra B-F. Spectrum B: fresh H₃PW₁₂O₄₀ sample after reduction by H₂; Spectrum C: H₃PW₁₂O₄₀ sample after reduction by pyrolysis products from HDPE; Spectrum D: H₂-reduced catalyst sample after three recycles and HDPE cracking; Spectrum E: H₂-reduced catalyst sample after five recycles and HDPE cracking. Spectrum F: sample obtained from re-oxidation of the Spectrum D sample in air with moisture at ambient temperature.

FIGS. 6A-6D illustrate representative reactions during thermal (FIG. 6A) and acid-catalyzed (FIGS. 6B-6D) pyrolysis.

FIG. 7. An illustrative up-flow fixed-bed reactor that may be used for catalyst pretreatment and polyolefin cracking according to the present methods.

FIG. 8. Gel permeation chromatograms of the polyethylene samples with 95% confidence bands.

### DETAILED DESCRIPTION

Methods for cracking a polyolefin are provided. In an embodiment, such a method comprises heating a blend comprising a polyolefin and an unsupported acidic polyoxometalate in a reaction zone of a reactor chamber, the reaction zone at a reaction temperature, while flowing a gas through the blend, to induce carbon-carbon (C—C) bond cleavage in the polyolefin to form a processed blend comprising cracked hydrocarbons. In the present disclosure, “C—C bond cleavage” and “cracked hydrocarbons” encompass products formed after C—C bond(s) are cleaved in the polyolefin as well as products formed from a variety of reactions that result from such cleavage (such as hydride and alkyl transfer reactions). The method further comprises passing at least a portion of the processed blend to a reflux zone of the reactor chamber, the reflux zone at a reflux temperature. Once in the reflux zone and depending upon the reflux temperature, some cracked hydrocarbons may pass through the reflux zone and be subsequently collected, while some cracked hydrocarbons may return to the reaction zone and undergo additional C—C bond cleavage. The process of passing at least a portion of the processed blend to the reflux zone in which some cracked hydrocarbons pass through and are subsequently collected, while some cracked hydrocarbons return to the reaction zone and undergo additional C—C bond cleavage may occur continuously while the reaction zone and the reflux zone are at their respective temperatures and while the gas is flowing. The C—C bond cleavage may also occur in the reflux zone. The method further comprises collecting the cracked hydrocarbons from the reaction zone, the reflux zone, or both.

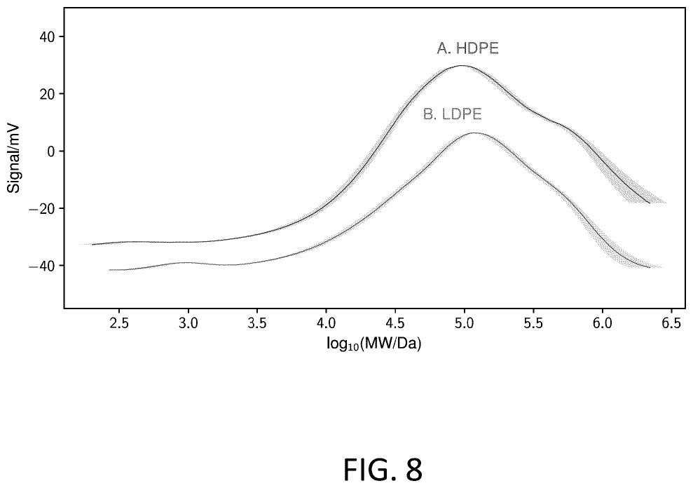

In the present disclosure, “polyolefin” refers to a class of hydrocarbon polymers which have been formed by polymerizing olefin monomers, such as α-olefin monomers. Illustrative polyolefins include polyethylene (polymerized ethylene), polypropylene (polymerized propylene), and polyisobutylene. The polyolefin may be characterized by its molecular weight and its degree of crystallinity. Regarding molecular weight, the polyolefin may have a number average molecular weight (Mn) in a range of from 1 to 10 kDa, from 10 to 100 kDa, from 100 to 600 kDa. This includes from 5 to 30 kDa, from 30 to 500 kDa, from 150 to 550 kDa, from 150 to 250 kDa, and a range formed using any of these values. The polyolefin may have a weight average molecular weight (Mw) in a range of from 500 kDa to 10,000 kDa. This includes from 1000 to 10,000 kDa, from 500 to 2000 kDa, and a range formed using any of these values. Both Mn and Mw may be measured as described in the Example below. (See also FIG. 8.) Regarding crystallinity, the polyolefin may be highly crystalline, having a crystallinity in a range of from 70% to 90%, including from 75% to 85%. Less crystalline polyolefins may be used, including those having a crystallinity in a range of from 15% to 35%, including from 20% to 30%. The polyolefin may be amorphous (i.e., no crystallinity) such as atactic polypropylene. If multiple types of polyolefins are used, they may have an average crystallinity in a range of 0% to 80%. Crystallinity may be measured as described in the Example below assuming linear transition in heat capacity. The polyolefin may be a homopolymer, but copolymers may be used, including a copolymer of ethylene and propylene. Illustrative polyolefins include high-density polyethylene (HDPE), low density polyethylene (LDPE), high-density polypropylene (HDPP), and low-density polypropylene (LDPP). A single type or a combination of different types of polyolefins may be used.

The polyolefin may be provided as pieces of a consumer product (e.g., a bottle, a container, etc.), whether or not that consumer product has been used by a consumer. Consumer products are distinguished from virgin polyolefin, which may be commercially available for use in a laboratory, but which has not been processed into a consumer product. As such, the consumer product polyolefin may comprise other components that may not be present in virgin polyolefin, e.g., polyolefin fragments, crosslinkers, crosslinker fragments, additives, dyes, etc.

The polyolefin features described above (molecular weight, degree of crystallinity, and source) distinguish the present methods from existing methods designed for cracking smaller molecular weight, less crystalline, and highly pure, laboratory grade, virgin polyolefins.

In the present disclosure, “acidic polyoxometalate” refers to the acidic form of a polyoxometalate, a class of strong solid Brønsted acids with high proton mobility. Desirably, the acidic polyoxometalate is highly soluble in water, an alcohol (e.g., methanol, ethanol), a carboxylic acid (e.g., formic acid, acetic acid), or combinations thereof. This includes having a solubility of at least 1 g/mL in one or more of such protic solvents. The acidic polyoxometalate may comprise W, Mo, V, or combinations thereof. Illustrative suitable acidic polyoxometalates include those represented by the formula HₓM₃₋ₓPM′_{y}W_{12-y}O₄₀, wherein M is an alkali metal (e.g., K, Rb, Cs, Li, Na), M′ is Mo or V, y is from 0 to 12, and x is from 0 to 3. Both x and y may be fractions. For example, H₃PW₁₂O₄₀ is an illustrative acidic polyoxometalate that may be used.

In the present methods, the acidic polyoxometalate is unsupported, by which it is meant that it is not combined with any support structure, e.g., is not immobilized on a surface of another material such as clay or a silicate (e.g., MCM-41, SBA-15). Thus, the blends being used in the present methods may be characterized as free of such support structures/materials. In this way, the acidic polyoxometalate is freely dispersed throughout the blend and freely diffusing during the present methods. This is unusual and contrary to existing approaches which considered that such support structures were necessary to induce cracking. (See, e.g., Jalil, Pasl A. Journal of Analytical and Applied Pyrolysis 65.2 (2002): 185-195.) The morphology of the unsupported acidic polyoxometalate in the present methods may be that of a plurality of solid particles, as noted above, dispersed throughout the blend. The relative amount of the polyolefin and the unsupported acidic polyoxometalate being used during the present methods may be adjusted to achieve a desired result, e.g., a certain overall yield of cracked hydrocarbons, a certain product distribution, etc. However, due to the ability of the unsupported acidic polyoxometalate to be recovered from the processed blend and reused as further described below, relatively large amounts may be used. This includes a weight ratio of polyolefin:unsupported acidic polyoxometalate of 1:1. The unsupported acidic polyoxometalate may be simply referred to as “catalyst.”

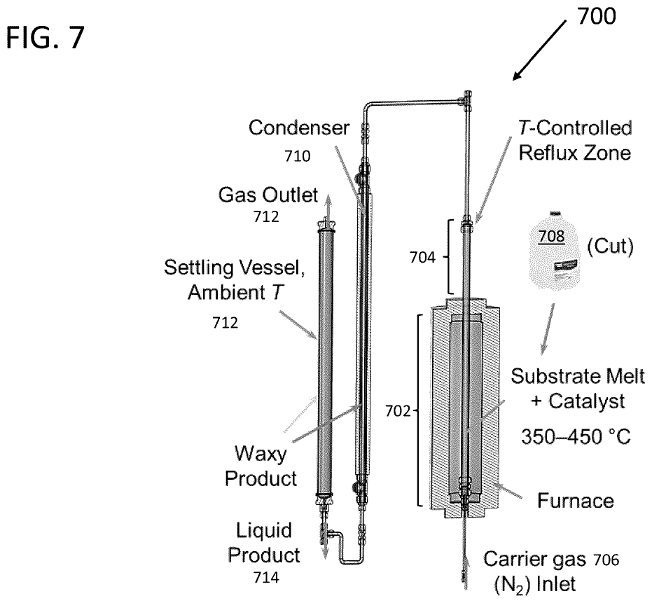

The present methods may be carried out in reactor systems such as the reactor system 700 shown in FIG. 7, comprising a reaction zone 702, a reflux zone 704 in fluid communication with the reaction zone 702, both configured to allow a gas 706 to be flowed therethrough. As shown in FIG. 7, both the reaction zone 702 and the reflux zone 704 are temperature controlled (independently) to achieve a desired reaction temperature and reflux temperature, respectively. The reaction zone 702 is also configured to contain the blend of the polyolefin 708 (here, pieces of a milk jug) and the unsupported acidic polyoxometalate (not shown) while the reflux zone 706 is configured to contain at least a portion of the processed blend.

Parameters such as the reaction temperature, reflux temperature, gas flow rate, and overall reaction time may be adjusted to achieve a desired result, e.g., certain overall yield of cracked hydrocarbons, a certain product distribution, etc. However, illustrative reaction temperatures include from 500 to 700 K or from 550 to 675 K (or a range using any of these values). The reaction temperature is generally sufficiently high so as to melt the polyolefin and sufficiently reduce its viscosity, resulting in the blend comprising molten polyolefin and solid, unsupported acidic polyoxometalate dispersed throughout. Illustrative reflux temperatures include from 300 to 475 K or from 325 to 450 K (or a range using any of these values). Generally, the reflux temperature is less than the reaction temperature so that some vaporized portions of the processed blend may cool in the reflux zone and return to the reaction zone wherein additional C—C bond cleavage and hydride/alkyl transfer reactions occur. As demonstrated in the Example, below, the particular combination of the reaction temperature and reflux temperature may be selected to achieve a specific product distribution. For example, for a given reaction temperature, higher reflux temperatures lead to a product distribution skewed towards higher molecular weight cracked hydrocarbons while lower reflux temperatures lead to a product distribution skewed towards lower molecular weight cracked hydrocarbons. Regarding the flowing gas, it may be composed of a single type of carrier gas, e.g., N₂, or a mixture of different carrier gases. Illustrative gas flow rates include from 100 to 500 std. cm³ min (sccm). Regarding overall reaction time, this refers to the length of time the reaction zone and the reflux zone are being heated at their selected reaction/reflux temperatures. Illustrative times include from 5 h to 24 hr or from 8 hr to 15 hr (or a range of between any of these values).

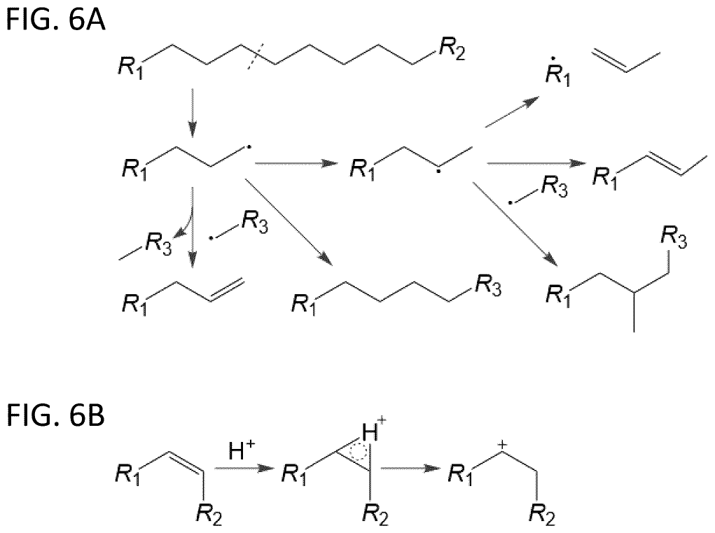

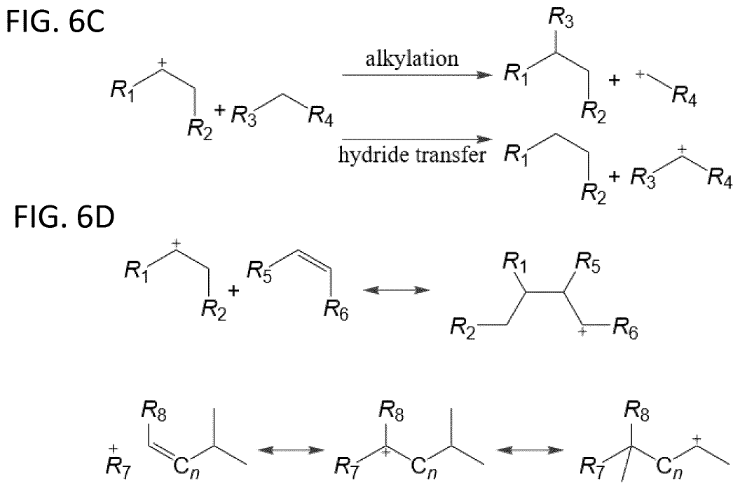

As noted above, the present methods include collecting the cracked hydrocarbons, including those generated from C—C bond cleavage occurring in the reaction zone, the reflux zone, or both. Such reactions are illustrated in FIGS. 6A-6D. Thus, the cracked hydrocarbons comprise a variety of hydrocarbons, including those that may be in their gas phase, liquid phase, solid phase, and generally, a combination thereof. As such, and as shown in FIG. 7, the reactor system 700 further comprises components configured to collect the various cracked hydrocarbons, including a condenser 710 and a settling vessel 712 (in both of which solid products may be collected), and outlets from which gaseous products (gas outlet 714) and liquid products (liquid outlet 716) may be collected. Solid products (e.g., coke) may also be present in the reactor chamber itself. These solid products may be separated from the unsupported acidic polyoxometalate as will be described below.

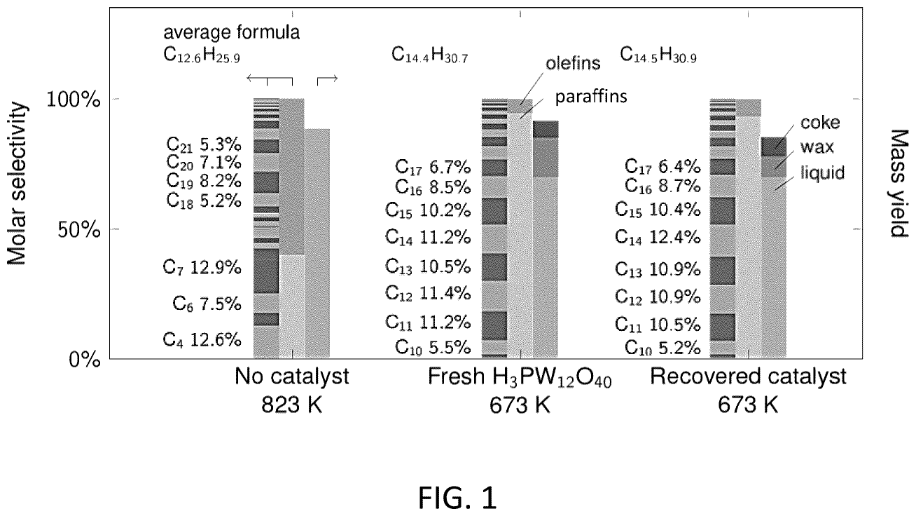

Further regarding the products (cracked hydrocarbons), the present methods may be characterized by an overall product yield and a product distribution. Overall product yield refers to a total weight of cracked hydrocarbons as compared to a total weight of polyolefin. As demonstrated in the Example, below, the present methods are able to achieve overall product yields of at least 80%, at least 85%, at least 90%, and at least 95%. (See also FIGS. 1, 2, and 4.) This includes a range of between any of these values. Product distribution refers to the type of collected hydrocarbons (e.g., number of carbon atoms in the hydrocarbon) and the amount of each type. Associated with the product distribution is an average molecular formula that may be calculated therefrom as described in the Example, below. For example, as shown in FIG. 1, the leftmost bar of the middle grouping labeled “Fresh H₃PW₁₂O₄₀” shows the distribution of products collected using this unsupported acidic polyoxometalate in the present method, shown as the mole percent of hydrocarbons having a certain number of carbon atoms (e.g., the products collected included 5.5 mol % of hydrocarbons containing 10 carbons). Above is shown the average molecular formula that corresponds to the measured product distribution, i.e., the average molecular formula of the collected cracked hydrocarbons is C_{14.4}H_{30.7}. The product distributions obtained using the present methods also show that substantially all of the cracked hydrocarbons are olefins and paraffins. This includes at least 80%, at least 85%, at least 90%, and at least 95% of the cracked hydrocarbons being olefins and paraffins.

As noted above, the present methods may further comprise recovering the unsupported acidic polyoxometalate from the reactor chamber after it has been used at least once and reusing it to carry out the present methods one or more additional times. This enables a continuous loop of polyolefin cracking and catalyst recovery. Recovery may be accomplished by exposing the used unsupported acidic polyoxometalate to any of the protic solvents described above (e.g., water, alcohol, carboxylic acid, or combinations thereof) to form a recovery solution comprising dissolved unsupported acidic polyoxometalate and any undissolved solid product (i.e., coke) that is generally also present; filtering the recovery solution to provide a filtrate; and precipitating unsupported acidic polyoxometalate from the filtrate. The precipitate (the unsupported acidic polyoxometalate) may be referenced via the descriptor “recovered” or “recycled.” As shown in FIG. 1, the overall product yield and product distribution using the recovered unsupported acidic polyoxometalate in the present methods is substantially the same as that of previously unused (“fresh”) unsupported acidic polyoxometalate. Not bound to a particular theory, it may be inferred from the results shown in the Example, below, that the cracking reactions on a fresh acidic polyoxometalate also compete with less dominant reactions involving reduction of the acidic polyoxometalate by the hydrocarbon species in the reactant and product as well as reactions that results in the formation of carbonaceous deposits.

Further regarding the unsupported acidic polyoxometalate, as demonstrated in the Example below, it was found that pre-reducing the unsupported acidic polyoxometalate prior to use in the present methods is useful as it, e.g., lowers the reaction temperature required to achieve a desired overall product yield/product distribution; reduces coking and thus facilitates recovery of the unsupported acidic polyoxometalate; and preserves the atomic structure (e.g., the Keggin structure) of the unsupported acidic polyoxometalate. Thus, the present methods may make use of pre-reduced unsupported acidic polyoxometalate.

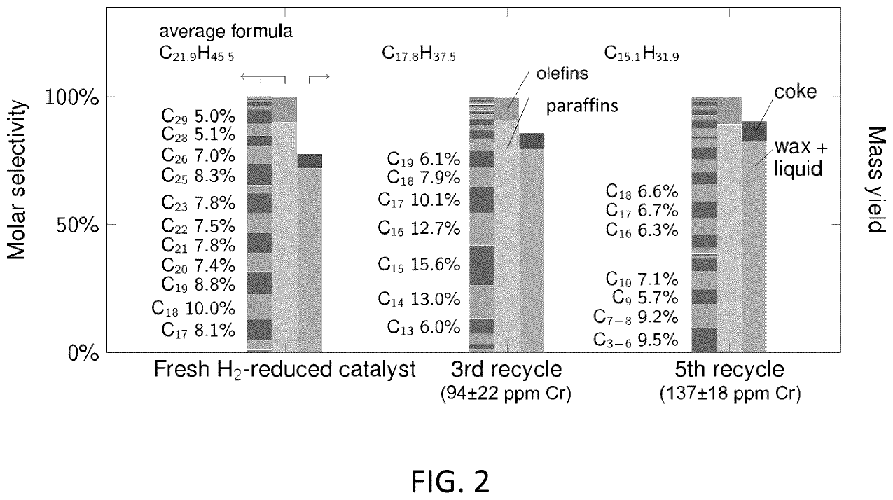

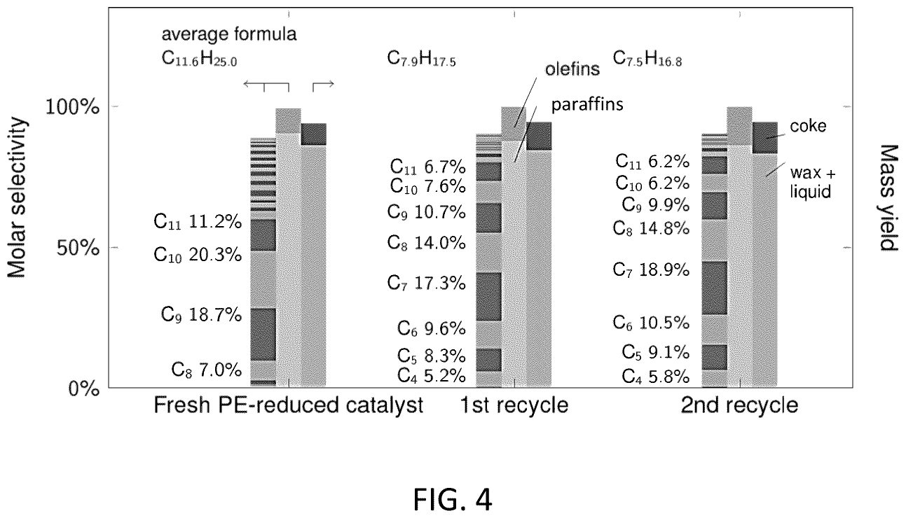

Pre-reduction may be carried out using the same reactor system for carrying out polyolefin cracking. Pre-reduction involves exposing the unsupported acidic polyoxometalate to a reductant while heating at a pre-reduction temperature for a pre-reduction time. The reductant refers to a chemical species capable of reducing the acidic polyoxometalate (i.e., donating an electron(s) to the acidic polyoxometalate). The reductant may be a gas and/or may be provided in a gas mixture with a carrier gas (e.g., N₂), e.g., by flowing the gas/gas mixture through the unsupported acidic polyoxometalate. The specific reductant, its amount, flow rate, the pre-reduction temperature, and the pre-reduction time may be selected to achieve a desired result e.g., overall yield of cracked hydrocarbons, a certain product distribution, etc. However, illustrative reductants include H₂, alcohols (e.g., methanol), aldehydes, alkanes (e.g., hexane), and olefins (e.g., butene). Olefins may be provided by cracked hydrocarbons derived from pyrolysis (e.g., thermal pyrolysis) of a selected polyolefin. That is, the products of polyolefin pyrolysis may also serve as suitable reductants. Comparing FIG. 2 (results for “fresh H₂-reduced catalyst”) to FIG. 4 (results for “fresh PE-reduced catalyst”) shows that using cracked hydrocarbons derived from the pyrolysis of HDPE as the reductant skews the product distribution to lighter cracked hydrocarbons and substantially lowers the average molecular formula thereof.

Regarding the pre-reduction temperature, illustrative values include any of those provided above for the reaction temperature. Regarding the pre-reduction time, illustrative times include from 1 to 5 hr, or from 2 hr to 3 hr. Regarding the amount of the reductant, illustrative values include from 0.2 vol % to 4 vol %, 0.2 vol % to 5 vol %, from 0.5 vol % to 2 vol % (balance carrier gas). Regarding flow rates, illustrative values include any of those provided above for catalytic pyrolysis with the unsupported acidic polyoxometalate.

The Example, below, further demonstrates that the unsupported acidic polyoxometalate may be doped with certain types of transition metals at certain amounts. This has been found to be useful to achieve a desired product distribution, including one skewed to lighter cracked hydrocarbons. (See 3^{rd} and 5^{th} recycle results in FIG. 2.) Thus, the present methods may make use of doped, unsupported acidic polyoxometalate. The dopant may be a transition metal such as Cr, Zr, Ti, Fe, or a combination thereof. The amount of the dopant is generally within a critical range so as to enhance cracking but without substantially disrupting the desirable (e.g., Keggin, Dawson, etc. and combinations thereof) structure of the acidic polyoxometalate. Illustrative amounts include at least 50 ppm or 100 ppm, but no more than 1500 ppm, no more than 1250 ppm, no more than 1000 ppm, or no more than 750 ppm. A range of between any of these values may be used, including a range of 20 to 1000 ppm. The amounts may be measured as described in the Example below. Since some used polyolefins may include any of the disclosed dopants (from the synthesis of such polyolefins), doping the unsupported acidic polyoxometalate may be accomplished by carrying out the present methods one or more times (e.g., two, three, or four times) to achieve the unsupported acidic polyoxometalate doped at the desired amount.

### EXAMPLE

Additional information regarding this Example, including data indicated as not being shown, may be found in U.S. Provisional Patent Application No. 63/642,991 that was filed May 6, 2024, the entire contents of which are hereby incorporated by reference.

### Introduction

This Example investigates the effects of catalyst pre-reduction and progressive Cr accumulation on catalytic cracking activity and recyclability. Specifically, the catalytic cracking of post-consumer HDPE samples into mixed hydrocarbons over W-based acidic polyoxometalates at ambient pressure and temperatures ≥573 K was investigated. A relatively simple method to separate the coke from the spent catalyst was demonstrated, wherein the catalyst was selectively dissolved in water and reprecipitated by drying the catalyst solution. As a carrier of reducing equivalents from the ethylene precursor of HDPE, Cr bonded strongly in the catalysts, sharing its d electrons. Progressive Cr incorporation into the catalyst with each recycle shifted the product distribution to lighter hydrocarbons. Such changes in product distribution were also observed when the catalyst was pre-reduced by a small amount of pyrolysis products (olefins) from HDPE. Electron paramagnetic resonance (EPR) spectroscopy of the various catalyst samples revealed a correlation between higher cracking activity and the presence of unpaired delocalized electrons, originating from either Cr(III) insertion or partial oxidation of olefins.

### Materials and Methods

### Materials

The nitrogen (high purity) was purchased from Matheson. The 12-tungstophospheric acid was purchased from Strem Chemicals. The high-density polyethylene (HDPE) materials (up to 0.7 mm thick) were from the containers of one gallon Walmart and Kroger branded distilled water. The 0.63 cm thick low-density polyethylene (LDPE) plate was purchased from McMaster-Carr (catalog number 8657K331). The plastics were cleaned with a metal brush and Alconox Detergent to remove labels and possible contaminants on the surface, and then washed with water and acetone. The samples were cut to about 1 cm×1 cm area and dried around 333-343 K to remove the acetone on the surface. No substantial differences could be observed from the FTIR spectra of the cleaned HDPE samples from the two sources.

### Reactor Unit and Reaction Procedure

Polyolefin cracking in a stirred batch reactor is challenged by transport limitations and accumulation of heavy hydrocarbons on the catalyst. Thus, an upflow fixed-bed reactor setup was tested, redesigned, and optimized for performing the reaction. A heated stream of inert gas (N₂) passing through the mixture of molten substrate (polyolefin) and solid catalyst could provide substantially better mixing and uniform heating compared to the Parr reactor. Further, the inert gas also serves to transport the cracked products from the reaction zone, allowing better control of product selectivity. The pyrolysis experiments were performed in a tubular reactor (FIG. 7). The tubular reactor contained stainless-steel ball bearings in the bottom zone, which preheated the incoming gas. A layer of glass fiber (˜3 mm thick) was placed above the bearings to retain the substrate and the catalyst above the preheat zone. The 1″ reaction section was mostly in the furnace, while its top part and the 1.27 cm (nominal ½ inch) section were both heated by heating tape when a hot reflux zone was needed. The effluent flow was first led through a 1.27 cm (nom. ½ inch) tubular condenser with counter-current coolant flow, and then led into a 5.08 cm (nom. 2 inch) settling vessel. Since the products had low surface tension and could be condensed as a mist, sufficient volume was required to collect all the liquid product. A gas sample could be collected at the outlet above the collection vessel. During the run, the condenser was set to 283 K in order to trap the products. At the end of a run, the condenser was set to 308-338 K so that the products flowed faster. The valve at the bottom of the collection vessel could be opened to collect the liquid products.

The catalyst was ground (particle size <0.3 mm), and the cleaned HDPE bottle (substrate) was cut into pieces that were around 1 cm² face area×0.7 mm thickness. For a typical run, the catalyst and the substrate were loaded above the glass fiber in the reactor, which was then assembled and situated in the furnace. The nitrogen flow was started at a desired rate, and the Variac that controlled the heating tape was set to a predetermined voltage for the desired reflux temperature. After ˜15-20 min stabilization, the furnace was ramped to the desired temperature at 10 K/min. After a desired reaction time when all the substrate had been cracked, the furnace was switched off while keeping the heating tape powered and the gas flowing. The heating and flow were turned off after the reactor cooled to room temperature.

When catalyst pre-reduction by hydrogen was performed prior to reactions, only the catalyst was loaded in the reactor. A purge flow of nitrogen at ˜100 cm³/min was started, the Variac was set to 35-40 V to heat the 1.27 cm (nom. ½ inch) tube above 373 K, and the furnace was set to ramp to the desired pretreatment temperature at 10 K/min. Once the temperatures stabilized, the nitrogen flow was changed to ˜80 cm³/min, and a flow of 4% hydrogen in nitrogen was started at ˜20 cm³/min. Following pretreatment for 2 h, the hydrogen/nitrogen flow was stopped, and the system was allowed to cool. With the nitrogen flowing, the reactor was opened at the joint of the 2.54 cm and 1.27 cm (nom. 1 and ½ inch, respectively) tubes, and the substrate was added into the reactor. The reaction with the pre-reduced catalyst was then started as described above.

For catalyst reduction by HDPE, the catalyst was loaded in a separate tubular reactor above HDPE isolated by a layer of glass wool to avoid direct contact leading to transfer of metal elements to the catalyst. The reduction was performed using a catalyst:HDPE mass ratio of 6:1 at 623 K for 4 h without reflux. Following the run, the reduced catalyst sample was separated from the coke (<0.05 g coke/g catalyst) as described in “Catalyst recovery,” below.

### Catalyst Recovery

The solid remnants in the reactor at the end of a run were collected and washed with chloroform to remove and analyze the absorbed hydrocarbons. They were generally in small quantities (<0.014 g products per g catalyst) and showed heavier carbon products than the collected liquid products. The washed solid was dried at 333-353 K.

The chloroform-washed solid was then added to distilled water (˜10 cm³ per g solid) and sonicated for up to 45 min to promote catalyst dissolution. The mixture was filtered using a ChemGlass filtration kit and polyvinylidene fluoride (PVDF) membranes with 0.1 μm pore size. To recover the solid catalyst, the solution was dried with either an oven (in air) or a rotary evaporator (under vacuum).

To recycle catalysts while maintaining their reduced forms, the spent catalyst was mixed with water that was double distilled to remove dissolved oxygen. The sonication and filtration were done in glassware with a balloon filled with argon to avoid exposure to ambient air. Finally, the blue filtrate was dried using a rotary evaporator at 333 K and 4 kPa with nitrogen backflush to maintain a low oxygen level. The solid was then further dried at ˜623 K in nitrogen for 4 h flowing at 100 std. cm³/min.

A mass flow chart (not shown) associated with the H₂-reduced catalysts during the recycling studies in FIG. 2 was obtained. The analysis shows that the strategy of dissolution and reprecipitation can recover at least 92%-96% catalyst in each run. A higher recovery rate for the overall process is expected when the losses due to transfer and quantification on a completely dehydrated basis are eliminated.

After the separation of coke and catalyst, the samples were packed in 4 mm ZrO₂ rotors for recording spectra on a Bruker Avance III 400 MHz spectrometer equipped with a Bruker 4 mm HX probe.

### Product Analysis

Following the reaction, the liquid products in the condenser and the collection vessel were allowed to settle at the bottom by gravity overnight. The collected liquid was dissolved in CHₓCl₄₋ₓ or CDₓCl₄₋ₓ for analysis by GC/FID and/or ¹H NMR spectroscopy. If the liquid nitrogen cooled condenser was used instead, a cold solvent was added to the condenser to maintain the products dissolved.

The GC analysis was carried out on an Agilent 7890A GC with a 100 m×0.25 μm×0.32 mm DB-PETRO column, with the column oven ramped from 303 K to 598 K at 10 K/min. When products in C₃-C₇ range were substantive, the ramping rate was reduced to 5 K/min for better separation in this range. The ¹H NMR spectra were collected on a Bruker Avance III HD 400 MHz spectrometer, using a 30° flip angle (zg30), relaxation delay of 1 s and 32 scans. The gas samples collected at the outlet of the collection vessel during a run were analyzed on the same Agilent GC, as well as on a Shimadzu GC with a ShinCarbon column connected to its TCD, using Ar as the carrier gas and a constant oven temperature of 303 K.

The product mixtures may have contained up to a few hundred compounds even below C₁₂ range (data not shown), among which some isomers with the same number of branching may coelute. Identification of carbon number, branching, and saturation for most products was done using the Kováts's retention indices (RI) of hydrocarbons compiled by ChemSpider (http://www.chemspider.com/). Linear alkanes (C₁-C₁₀, C₁₂, C₁₄, C₁₆), isobutane, propylene, butylene from Matheson, gasoline, and Jet Fuel A from Restek were used as authentic samples.

$${RI}_{i} = 100 [ n + \frac{log {t}_{i}^{′} - log {t}_{n}^{′}}{log {t}_{n + 1}^{′} - log {t}_{n}^{′}} ]$$

where RIᵢ is the retention index of peak i; tᵢ is the carbon number of normal alkane CₙH₂ₙ₊₂ eluting before peak i; tᵢ is the adjusted retention time for peak i; An adjusted retention time is retention time of a peak minus retention time of an unretained peak, tᵢ′=tᵢ−t₀; tₙ is the adjusted retention time for normal alkane CₙH₂ₙ₊₂.

Although retention indices for most hydrocarbons ≥C₁₂ are not available, the products associated with each carbon number ≥C₇ show similar patterns within each group in this Example. Thus, the identification can be extrapolated to heavy products.

### Characterization of the Polyethylene Substrates

### Differential Scanning Calorimetry (DSC)

The crystallinity of polyolefin is a crucial descriptor that correlates with its chemical structure (distribution of primary, secondary, tertiary carbons), and thereby to the intrinsic reactivity, viscosity, etc., in its molten state. Differential scanning calorimetry experiments for polyethylene samples were performed on a TA Instruments SDT Q600. For calibration of heat flow, an experiment was run with a sapphire standard from 294 K to 523 K, and the segment from 308 K to 523 K was used for calibration. The polyethylene samples were stabilized at 298 K for 15 min prior to ramping to 520 K at 10 K/min. The temperature and heat flow over time were recorded (data not shown).

The partial derivative of heat uptake (q) with respect to temperature (T) was calculated by dividing the heat flow (\dot{q}) by the numerical derivative of temperature with respect to time:

$$\frac{\partial q}{\partial T} = \frac{\dot{q}}{\dot{T}} .$$

The heat of phase change was estimated assuming a linear correlation between the heat capacities of the two phases in the temperature range of the phase transition. This method was also verified by assuming an ideal mixture heat capacity, cₘᵢₓ(T)=(1−x_{B}(T))×c_{A}+x_{B} (T)×c_{B}, and solving the following boundary value problem.

$$Δ H \frac{\partial {x}_{B}}{\partial T} + {c}_{mix} ( T ) = Δ H \frac{\partial {x}_{B}}{\partial T} + ( 1 - {x}_{B} ( T ) ) \times {c}_{A} + {x}_{B} ( T ) \times {c}_{B} = \frac{\partial q}{\partial T}$$

where A and B represent the initial and final phases as temperature rises, x_{B} represents the fraction of phase B, c_{A} and c_{B} [J/(g·K)] represent the heat capacities of the respective phases. The heat of phase transition is the fractional crystallinity times the heat of a crystalline polyolefin: ΔH_{crystal}=290 KJ/g, i.e., ΔH=f×ΔH_{crystal}.

The boundary conditions are:

$${x}_{B} ( T ) = 0 , T \leq {T}_{A} ; {x}_{B} ( T ) = 1 , T \geq {T}_{B}$$

where T_{A} and T_{B} represent the temperature range of the phase transition.

The curves of heat uptake and assumed heat capacity are not shown. AH was estimated by numerically integrating the area enclosed by the curves. The estimated crystallinity values were 82.9% for HDPE and 27.3% for LDPE (assuming linear transition in heat capacity) and 81.7% for HDPE and 27.8% for LDPE (assuming ideal mixture heat capacity).

### High-Temperature Gel Permeation Chromatography (HT-GPC)

High-temperature gel permeation chromatography (HT-GPC) of the polyethylene substrates was performed by by Poly Analytik Inc., using a Tosoh HLC-8321GPC/HT instrument equipped with a refraction index detector, following an established protocol for polyolefin analysis. The polydispersity indices (PDIs) estimated from the chromatograms of the polystyrene calibration standards [log₁₀(MW/Da) between 2.91 and 5.94] were between 1.07 and 1.20, indicating that extra instrumental broadening was insignificant. The molecular weight distributions of the HDPE and LDPE substrates were recorded and are shown in FIG. 8, with the 95% confidence bands reflecting the experimental measurement uncertainty based on the calibration curve.

Each of the nominal molecular weight distributions can be fitted to two Gaussian functions with the following μ and σ values:

$$HDPE : {μ}_{1} = 4.84 \pm 0.003 , {σ}_{1} = 0.37 \pm 0.003 , {μ}_{2} = 5.22 \pm 0.002 , {σ}_{2} = 0.76 \pm 0.003 .$$

$$LDPE : {μ}_{1} = 5.17 \pm 0.002 , {σ}_{1} = 0.36 \pm 0.004 , {μ}_{2} = 5.76 \pm 0.003 , {σ}_{2} = 0.21 \pm 0.003 .$$

The nominal values and asymmetric 95% confidence intervals of the overall weight-averaged molecular weight (M_{w}) and the number-averaged molecular weight (Mₙ) can be estimated from FIG. 8.

$${M}_{n} = \frac{\int VM ( t ) 𝕕 t}{\int V 𝕕 t} , {M}_{w} = \frac{\int {VM}^{2} ( t ) 𝕕 t}{\int VM ( t ) 𝕕 t}$$

where V is the detector signal, and M(t) represents the calibration curve correlating the molecular weights to the retention times. The estimates are shown in the Table below.

TABLE 1
Overall number-averaged molecular weights (Mₙ) and
weight-averaged molecular weights (M_{w}) of the polyethylene
substrates (with 95% confidence intervals).

| Sample | Mₙ | M_{w} |
|---|---|---|
| HDPE | 260₋₁₀₆⁺³¹⁶ | 3.31_{-1.87}^{+6.02} |
| | kDa | MDa |
| LDPE | 209₋₁₉⁺²⁰ | 636₋₉₂⁺³⁹² |
| | kDa | kDa |

### Characterization of Polyethylene Samples

Details of differential scanning calorimetry (DSC) for estimating fractional crystallinity and high-temperature gel permeation chromatography (HT-GPC) analyses for determining molecular weight distribution of the polyethylene substrates have been provided above.

### Catalytic Reactor and Operating Procedure

Preliminary experiments were carried out in a Parr reactor in the batch mode to establish feasibility of cracking HDPE with the polyoxometalate acid catalyst (data not shown). Details of the catalytic reactor including a schematic and operation details have been provided above. To better control the product distribution, the reaction was carried out in a continuous up-flow fixed-bed reactor loaded with polyolefin chips (˜1 cm²×0.7 mm thick) and catalyst powder (particle size <0.3 mm). The reactor was heated in an electric furnace. The heated inlet gas flow served to heat the reactor contents uniformly, eventually melting the polyolefin. The furnace was ramped to the desired temperature at 10 K/min. When catalyst pre-reduction by hydrogen was performed, only the catalyst was loaded in the reactor and reduced in a flowing H₂/N₂ stream at the desired pretreatment temperature. For catalyst reduction by HDPE product vapors by thermal cracking, the catalyst was loaded in a separate tubular reactor above a HDPE layer isolated by a layer of glass wool. In the catalytic cracking runs, the pretreated catalyst and the polyolefin chips were randomly mixed and loaded in the reactor. The volatile products were carried by the nitrogen gas from the reaction zone to the temperature-controlled exit section of the up-flow reactor, where heavier products were refluxed back to the reaction zone. The lighter fraction of the products was condensed and collected further downstream of the reactor.

### Catalyst Recovery

Following reaction, the spent catalyst was removed and worked up as described above. Briefly, the spent catalyst was washed with chloroform to remove any absorbed hydrocarbons, followed by drying. Double-distilled water was then added to the dried catalyst, and the slurry was sonicated to dissolve the catalyst. To avoid oxidation of the catalysts, the water was double distilled to remove dissolved oxygen. The sonication and filtration were done in glassware connected to an argon-filled balloon to avoid exposure to ambient air. The filtrate was dried in a rotary evaporator with nitrogen backflush to maintain a low oxygen level.

### Analysis of Cracked Products

Details of product analysis have been provided above. Briefly, following reaction, the liquid products were dissolved in CHₓCl₄₋ₓ or CDₓCl₄₋ₓ for analysis by GC/FID and/or ¹H NMR spectroscopy. The GC analysis was performed in an Agilent 7890A GC equipped with a DB-PETRO column. Gas phase samples were also analyzed by GC. The product mixtures contained up to several hundred compounds. The ¹H NMR spectra were collected on a Bruker Avance III HD 400 MHZ NMR spectrometer.

### Coke and Catalyst Characterization

Thermogravimetric analysis (TGA) and solid state ¹³C NMR spectroscopy of the solid products on the spent catalyst were performed on an SDT-Q600 TA Instrument and Bruker Avance III HD 400 MHz NMR spectrometer, respectively. Details of analysis along with sample spectra are not shown here.

Titration methods were employed to determine the acid density in dissolved samples of the fresh and recovered/reprecipitated catalyst samples. Procedural details and results are not shown here. Solid-state ¹H and ³¹P NMR spectra of these samples were also performed to discern structural changes in the catalyst (data not shown). Electron paramagnetic resonance (EPR) spectra of the fresh and recycled catalysts were collected on a Bruker EMXplus spectrometer equipped with an Oxford Instruments cryostat using X-band microwave. Details of EPR spectral simulation and fitting are not shown here.

### X-Ray Diffraction (XRD)

To prepare XRD samples, three samples containing various mass fractions of Cr were prepared as follows. A Cr(NO₃)₃·9H₂O precursor was dissolved in water, mixed with a solution containing excess disodium ethylenediaminetetraacetate, and reverse-titrated by barium nitrate using Eriochrome Black T as the indicator, to determine that its Cr(NO₃)₃·9H₂O content was 85.5%. A stock solution of the precursor was combined with a solution of H₃PW₁₂O₄₀·xH₂O (amounts as shown in Table 2, below) to provide Cr doped catalysts corresponding to the Cr/H₃PW₁₂O₄₀ mass fractions shown in Table 2, below. The mixtures were dried in an oven at ˜333 K overnight (15 h), calcined at 673 K in flowing zero grade air at 100 std. cm³/min for 4 h, and reduced at 623 K in a 0.8% H₂/N₂ stream flowing at 100 cm³/min for 2 h.

TABLE 2
Masses during doping Cr in H₃PW₁₂O₄₀
and the respective mass fractions

| | | | | Cr mass |
|---|---|---|---|---|
| | | | | fraction |
| Cr from | | | | (dehydrated |
| stock solution | | H₃PW₁₂O₄₀•yH₂O | | basis) |
| 10.24 mg | (0.197 mmol) | 12.0082 g | (3.892 mmol) | 914 ppm |
| 2.47 mg | (0.048 mmol) | 7.1846 g | (2.329 mmol) | 368 ppm |
| 3.86 mg | (0.074 mmol) | 6.5029 g | (2.108 mmol) | 636 ppm |

The powder XRD experiments were performed in a Panalytical Empyrean diffractometer. The experiments were completed within 30 min for samples that were transferred out of an inert environment. The incident beam was generated by an Empyrean Cu X-ray tube with a tension of 45 kV and a current of 40 mA. The 2θ angle resolution was 0.01°. The results were processed using the General Structure Analysis System (GSAS) II (https://github.com/AdvancedPhotonSource/GSAS-II.git, commit 1b33e96), following the instructions for background fitting, fitting and indexing in its tutorials (https://github.com/AdvancedPhotonSource/GSAS-II-tutorials.git, commit 8169f89).

### Results and Discussions

### Thermal Pyrolysis

Experiments were performed in an upflow tubular reactor with ˜10 g HDPE and a carrier gas (N₂) flow of 300 std. cm³/min. A preheated stream of flowing nitrogen uniformly heated and melted the polyolefin chips in the tubular reactor. In benchmarking thermal pyrolysis experiments, small quantities of olefins (C₂-C₆) were present in the effluent gas during thermal degradation of HDPE at 573, 623, and 673 K. Most of the substrates degraded into a brown solid or wax that remained in the tubular reactor. Thermal pyrolysis at 823 K produced a mixture of C₃-C₂₀₊ paraffins and olefins within 6 h (FIG. 1).

### Catalytic Pyrolysis

Catalytic cracking experiments were carried out with ˜10 g HDPE, ˜10 g 12-tungstophosphoric acid (H₃PW₁₂O₄₀), and a carrier gas (N₂) flow of 300 std. cm³/min. Although this is a high catalyst/substrate ratio, as will be discussed below, in this Example, the spent catalyst may be recovered and reused multiple times to process more substrate. FIG. 1 shows details of the product distribution from the cracking runs in three-column bar charts. The first column shows the carbon number distribution in the liquid+wax products. The second column shows the selectivity distribution by functional groups, predominantly paraffins and olefins with aromatics being negligible. The third column shows the mass yields of liquid, wax, and coke relative to the mass of the HDPE processed. Number-averaged formulae of products are shown above each group.

Table 3 summarizes how the reflux zone temperatures control the product distribution. At the lower reaction temperatures of 573 and 623 K, the extent of cracking is such that the major products were mostly heavy waxes when the reflux zone temperature was maintained at 403±10 K. When the reaction temperature was increased to 673 K at the same reflux zone temperature, the increased cracking rate yielded ˜70% as liquid products and ˜15% as waxes. The vapor stream contained minor amounts of C₃-C₁₈ (data not shown). The major products (average formula: C_{14.4}H_{30.7}) were branched paraffins in the C₈-C₃₂ range (FIG. 1), along with a small fraction of olefins. ¹H NMR spectra (data not shown) indicated relatively small fractions of olefinic (<10%) and aromatic protons (≤0.1%).

TABLE 3
Tunability of product distribution by varying reflux
temperatures at a reaction temperature of 673 K.

| | | |
|---|---|---|
| Reflux | Product yields, % | Average formula |

| T, K | Liquid | Wax + coke | of liquid products |
|---|---|---|---|
| 353 | 77.3 | 14.9 | C_{13.9}H_{29.4} |
| 403 | 70.0 | 21.7 | C_{14.4}H_{30.7} |
| 453 | ≥10.7 | — | — |

Conditions: HDPE loading: ˜10 g, catalyst loading: ˜10 g, carrier gas (N₂) flow: 300 std. cm³/min, run time: 16 h.

When the reflux zone temperature was increased to ˜453 K at the same reaction temperature (673 K), more of the heavier products entered the condenser, depositing as wax and decreasing the liquid product yield to ˜11 wt. % of the HDPE mass (data not shown). In contrast, when the reflux temperature was decreased to 353 K, more of the cracked products were returned to the reaction zone, shifting the hydrocarbon product distribution to lighter compounds and was more evenly distributed compared to the run with a reflux temperature of 403±10 K (FIG. 1). These results show that unsupported 12-tungstophosphoric acid catalysts can effectively crack polyolefin waste to produce a hydrocarbon crude consisting of mostly paraffins in a desired carbon number distribution.

### Cracking Mechanisms

FIGS. 6A-6D show representative reactions during thermal and acid-catalyzed pyrolysis involving paraffins and olefins. Although not directly observed, radical reactions (FIG. 6A) have been proposed to occur during thermal pyrolysis, including random scission, beta-scission, and hydrogen transfer. These reactions can happen intermolecularly or intramolecularly and at any position of a chain.

The C═C bonds produced from the thermal radical reactions can react with acidic protons to produce the initial carbonium ions (FIG. 6B). As illustrated in FIGS. 6C and 6D, these intermediates can cleave a C—C⁺ bond and/or undergo a variety of intermolecular and intramolecular transfer of fragments such as hydride, methyl, and larger alkyl/alkenyl groups. Alkylation, hydride transfer, isomerization, oligomerization, and scission reactions are known to produce a diverse pool of olefins and paraffins, as well as carbonium ions, in olefin-paraffin alkylation. Similarly, the carbonium ions may attack any arbitrary position in any carbon chain and reduce the average molecular weight of heavy hydrocarbons. Despite similarity in mechanisms, it is noted the present methods involve a reduction in carbon number of the products which is in sharp contrast to alkylation reactions wherein an increase in the alkylate product carbon number is desired.

### Recovery and Reuse of Spent Catalyst

Following the reaction runs using fresh catalyst, the catalyst was recovered by washing the solid remnants in the reactor with double-distilled water, sonicating the wash for 45 min, filtering the solids, and drying them in an oven at ˜333-343 K. Approximately, 74-89% of the initial catalyst mass was recovered by reprecipitation of the filtrate. The recovered catalysts from various runs were loaded into the reactor, along with reduced amounts of HDPE (˜9 g to maintain a 1:1 mass ratio of substrate and catalyst) to evaluate their activity at 673 K with reflux at 403±10 K. The product distribution obtained was almost similar to that observed with the fresh catalyst (FIG. 1). However, ≤2.7 g of the acid catalyst could be recovered after the first recycle run, following an identical catalyst recovery procedure as before. This suggests that, unlike the run with the fresh catalyst, the polyanions may be encapsulated within the coke in the recycle runs, making their dissolution and separation more difficult. To better understand this phenomenon, a run with a substrate:catalyst ratio of 1:3 was performed at a reactor temperature of 673 K and a reflux temperature of 403±10 K (data not shown). While the product distribution was similar to previous runs (data not shown), a blue aqueous phase was evident when the spent catalyst was sonicated with distilled water for 8 minutes (data not shown). This indicates the presence of reduced polyoxometalates (referred to as “heteropoly blue”). Interestingly, the mass yield for coke increased to ˜33%. This suggests an additional coke formation pathway via substrate dehydrogenation to reduce the polyoxometalates. Following separation of the catalyst, analysis (data not shown) of the solid remnants revealed the formation of typical aromatic coke that can be completely burned in air above 770 K.

At lower temperatures (<373 K), polyoxometalates such as tungstates with iron, vanadium, or molybdenum heteroatoms, as well as molybdates (HNₓM₃₋ₓPMo_{y}W_{12-y}O₄₀ and HₓM₃₋ₓPV_{y}W_{12-y}O₄₀, where M is an alkali metal) can oxidize C₁-C₄ alkanes in the presence of an oxidant such as H₂O₂ and N₂O. However, 12-tungstophosphoric acid shows very low oxidation activity. The results described in this Example suggest that 12-tungstophosphoric acid is more susceptible to reduction at higher temperatures by dehydrogenation of aliphatic substrates. It was therefore hypothesized that catalyst pre-reduction would be useful to minimize substrate dehydrogenation and the resulting coke formation.

Cracking with Hydrogen-Reduced Catalyst

The H₃PW₁₂O₄₀ catalyst (10 g) was pre-reduced at 623 K in a 0.8% H₂/N₂ stream flowing at 100 std. cm³/min for 2 h. It was found that hydrogen pretreatment promoted cracking activity at lower temperatures (as low as 573 K) compared to the case without the reductive pretreatment. However, the extent of cracking was less at 573 K and produced mostly wax even when the reflux temperature was varied from 313-403 K. Increasing the reactor temperature to 623 K with a reflux temperature of 403 K still produced mostly waxy products (melting point >338 K). However, when the reflux temperature was reduced to 313 K at the same reactor temperature (623 K), the increased refluxing allowed more of the primary products to undergo repeated cracking, resulting in lighter hydrocarbons with a melting point around 308 K and an average formula of C_{21.9}H_{45.5} (FIG. 2). Consistent with the inventors' hypothesis, hydrogen pretreatment of the catalyst did reduce coke formation on the spent catalysts such that they could more easily recovered and recycled.

As described above, catalyst recovery was performed, taking precaution to minimize reoxidation by ambient air. Prior to each recycle run, the recovered catalyst was pretreated in flowing 0.8% H₂/N₂ mixture for 1 h to reduce any W species that might have been oxidized during catalyst recovery. The product distribution progressively shifted to lighter hydrocarbons with each recycle run (FIG. 2). This necessitated the use of a short tube cooled by liquid nitrogen (instead of the tubular condenser) to provide sufficient cooling and more completely recover the lighter products. The catalyst recovery increased to 92%-96% during the recycle runs (data not shown). By the fifth recycle, the yield of C₃-C₆ hydrocarbons, which were not dominant in the run with the fresh catalyst, substantially increased. Such a shift in carbon number distribution with each recycle run led to speculation that metal impurities present in HDPE may have been accumulating in the recovered catalysts and influencing their activity. For example, metals (such as Ti, Zr, Cr, and Fe) in typical polymerization catalysts are present as residues in polymer products. Inductively coupled plasma optical emission spectroscopy (ICP-OES) analysis of the catalyst samples recovered after the third and the fifth recycle runs revealed Cr mass concentrations of around 94±22 ppm and 137±18 ppm, respectively (see FIG. 2 and other data not shown data not shown).

### Effects of Cr Incorporation on Catalyst Structure

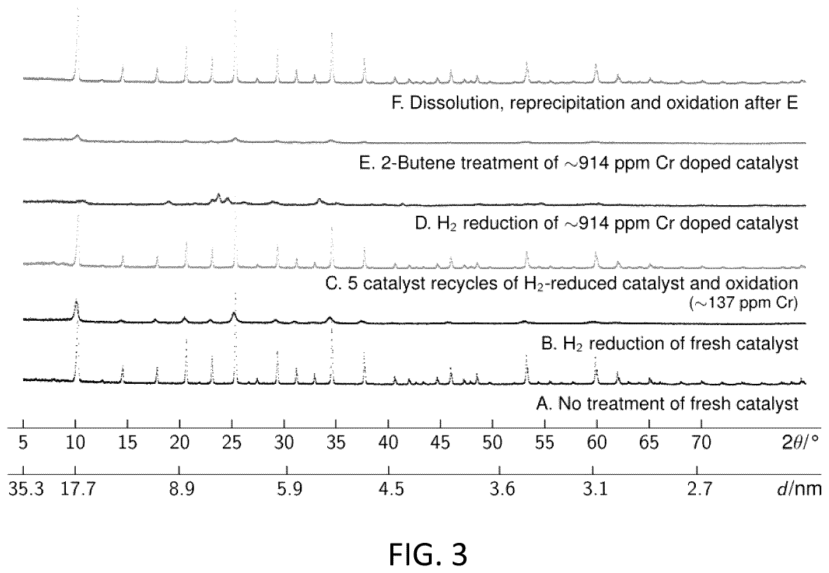

The structural effects of H₂ pre-reduction on the Cr-containing catalyst samples were studied by X-ray diffraction experiments and analysis. The XRD results show that H₂ reduction at 623 K induced oxygen defects in H₃PW₁₂O₄₀ (FIG. 3, Spectrum B). When the catalyst was recovered after five recycles (containing ˜137 ppm Cr) and re-oxidized, the XRD spectrum (FIG. 3, Spectrum C) was similar to that of the fresh catalyst (FIG. 3, Spectrum A), suggesting that the cage structure was preserved despite Cr incorporation. However, when the H₃PW₁₂O₄₀ catalyst was intentionally doped with ˜914 ppm Cr and reduced with H₂, the XRD signature (FIG. 3, Spectrum D) was similar to that of oxide phase(s) of tungsten, indicating structural disintegration of the catalyst. It was also observed that this catalyst was only partially soluble in water (data not shown). As a weaker reductant than hydrogen, 2-butene was evaluated (as a model olefin for the pyrolysis products) and found to preserve the cage-like structures better even with 914 ppm Cr incorporation (FIG. 3, Spectra E and F). Hence, the effect of reduction by the HDPE pyrolysis products on cracking activity was further evaluated.

Catalytic Cracking with Hydrocarbon-Reduced Catalyst

Unlike hydrogen that allows only two-electron transfer, products from polyolefin cracking can be partially oxidized to form charged and/or radical fragments. Further, the incorporation of the Cr that has been reduced by the ethylene precursor during polyethylene synthesis also involves electron transfer to the cage-like structures, essentially reducing the catalysts. Given these possibilities and the observation that olefin pre-reduction better preserved the catalyst structure, the products from HDPE cracking were evaluated as reductants. To avoid direct contact between the catalyst sample and HDPE during the reduction step, the H₃PW₁₂O₄₀ was loaded in the up-flow tubular reactor above the HDPE chips with a layer of glass wool separating the two sections. The reduction was performed using a catalyst:HDPE mass ratio of 6:1 at 623 K for 4 h without reflux. Following the run, the reduced catalyst sample was separated from the coke (<0.05 g coke/g catalyst) and recovered as described above.

As shown in FIG. 4, runs with hydrocarbon-reduced catalysts produced paraffins, olefins, and coke, similar to runs using H₂-reduced catalysts. However, lighter products (<C₈) were observed with the catalyst even without Cr accumulation (FIG. 4). During the next two cycles with recovered catalyst, the production of lighter products further increased. The minor increase in the Brønsted acid density of the recycled catalysts (≤12%, data not shown) cannot solely explain such an increase in the cracking activity of the catalysts. The increased cracking activity is likely also related to the reduction of polyoxometalates and acid strength.

Since olefins from PE pyrolysis are weaker but more versatile reductants than H₂, each polyoxometalate cluster likely underwent reduction with fewer electron transfers than those reduced by H₂. The proton mobility of polyoxometalates could possibly have been increased by the presence of extra delocalized electrons. Further, the observations from XRD experiments (FIG. 3 and other data not shown) clearly demonstrated that reduction using olefins was less destructive than reduction using hydrogen. Although such redox characteristics of acidic polyoxometalates do not manifest in their applications at relatively low temperatures, they may contribute toward extra coke formation in other applications at high temperatures partly via increased acidity and partly via dehydrogenation as well. As observed in the case of hydrogen-reduced catalysts, Cr accumulated in the recovered catalyst with each recycle and partially contributed to the enhanced cracking activity as inferred from the decreasing average molecular formula of the hydrocarbon products. Cr presence in these recovered catalysts was confirmed qualitatively via ICP-OES.

The foregoing results demonstrate that acidic polyoxometalates can effectively crack highly crystalline HDPE. The less crystalline low-density polyethylene (LDPE) with better transport properties also underwent facile cracking with polyoxometalates (data not shown). The cracked products of LDPE exhibited a lower carbon number distribution, highlighting the correlation between polyolefin structure, crystallinity, and their case of deconstruction.

Insights into Catalyst Reduction Effects by Electron Paramagnetic Resonance (EPR) Spectroscopy

Polyoxometalates showed interesting redox characteristics. For example, electrochemical one-electron and two-electron reductions of [PW₁₂O₄₀]³⁻ to [PW₁₂O₄₀]⁴⁻ and [PW₁₂O₄₀]⁵⁻, respectively, have been demonstrated in aqueous solutions. However, during the reduction of polyoxometalates in their solid state in this Example, the detection of water indicates that oxygen must be lost from the Keggin cages since external oxygen interference was carefully avoided in the experiments. Such changes were likely related to the increased activity of the reduced catalyst despite minor changes in acid density determined by reverse titration (data not shown). Likely due to the delocalized electronic bands (data not shown), polytungstates are more readily reduced and oxidized either thermally or electrochemically, and are excellent electron reservoirs. In contrast, only at higher temperatures (>500° C.) can bulk W^{VI}O₃ be reduced to intermediate oxide phases, such as W₁₈O₄₉ and W₄O₁₁, by various reductants prior to reduction to W^{IV}O₂.

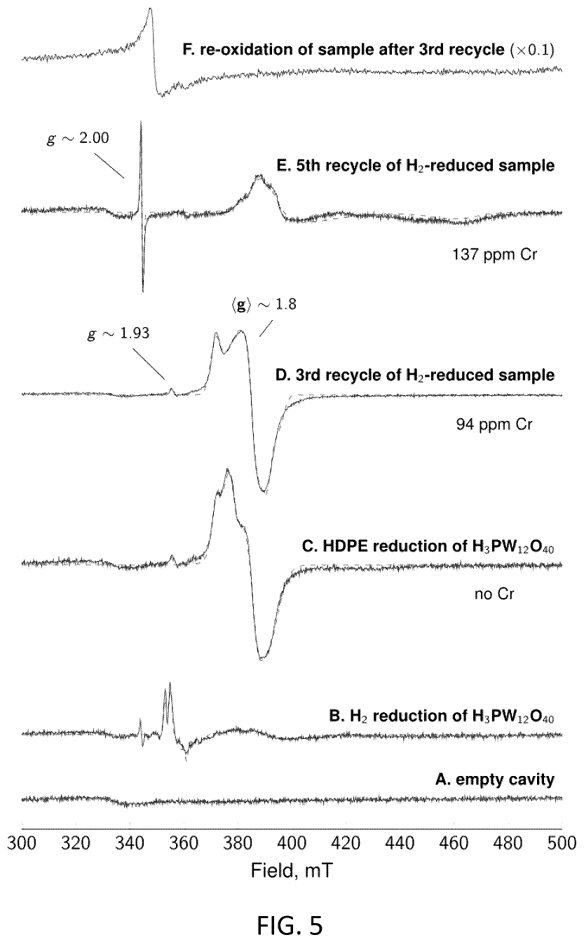

In the EPR spectra of reduced catalyst samples, isotropic (represented by the degenerate g values around 2.00 and 1.93) and anisotropic signals (represented by the means of g values, <g>, around 1.8) were generally observed (FIG. 5, Spectra A-F). No hyperfine coupling could be observed, which is consistent with computational results that only 170 (however, negligible natural abundance of 0.0380%) may contribute substantially to A tensors due to the electron densities being closer to the O atoms. For the sample immediately after H₂ reduction (FIG. 5, Spectrum B), weak resonances of g˜2.00 spins were evident, similar to electrons trapped by oxygen vacancies and reduced metal centers. The weak signal intensity near g˜1.8 suggests that H₂ reduction likely introduced an even number of electrons per polyoxometalate cluster on average, resulting in a diamagnetic ground state. Further, calculations showed that during two-electron reduction, loss of a bridging oxygen from the cage structure was preferred over loss of a non-bridging oxygen (data not shown). Also, the ground state for either case had a multiplicity of 1, suggesting that the delocalized electrons of the polyoxometalates were mostly paired.

The EPR spectrum corresponding to the H₂-reduced catalyst sample recovered after the third recycle run (FIG. 5, Spectrum D) shows an anisotropic signal [<g>˜1.8], and the one after the fifth recycle (FIG. 5, Spectrum E) shows an isotropic signal [g˜2.00] and an anisotropic signal [<g>˜1.8]. The signal corresponding to g˜2.00 after the fifth recycle (FIG. 5, Spectrum E) was more intense than the similar one from the fresh H₂-reduced sample (FIG. 5, Spectrum B), likely due to incorporation of CrOₓ inducing more defects, leading to broken symmetry in the electronic structures. The Cr was likely introduced as Cr(III), since the Cr in olefin polymerization catalysts usually undergoes reduction from Cr(VI) oxides by the olefins. The aqueous standard electrode potentials for Cr²⁺ and Cr³⁺, as well as the photocatalytic reduction of Cr(VI) by polyoxometalates, also suggest that the Cr was likely introduced at a +3 oxidation state. As such, each Cr(III) introduced an odd number of electrons into the molecular orbitals (MOs). The change in resonances observed from the third to the fifth recycle for the catalyst samples suggests multiple possible locations of Cr incorporation and structural change. Meanwhile the Cr orbitals partially hybridized (data not shown) into lower-energy MOs localized near the Cr atoms (g values around 1.9-2.0) and higher-energy MOs delocalized over the Keggin structure (g values around 1.7-1.8). This inhomogeneity may be also observed from the skewed intense signal in FIG. 5, Spectrum F, which could be related to oxygen anions and Cr species, after the sample corresponding to FIG. 5, Spectrum D was re-oxidized in air with moisture at ambient temperature. The shift of resonances likely suggests broken symmetry also in the electronic structures by Cr introduction, as illustrated by the computation results (data not shown), as well as the changes in optical absorption.

The signals corresponding to <g>˜1.8 spins indicate that the unpaired electron was highly delocalized. However, their anisotropy appeared different from the nearly isotropic signals from [PW₁₂O₄₀]⁴⁻ (from one-electron reduction of [PW₁₂O₄₀]³⁻) and other one-electron reduced hetero-12-tungstates. The aqueous electrochemical reduction potential of such polyoxometalates shows a dependency of reducibility on structure and heteroatom. Hence, Cr likely changed the reducibility of the polyoxometalate. Cr may participate in alternative polyoxometalate structures, join heteropolytungstates into larger units, or cap heteropolytungstates. Lower symmetry of such structures than that of α-[PW₁₂P₄₀]³⁻ (T_{d}) could be related to anisotropic g tensor. However, the XRD patterns (data not shown) support Cr substitution of W that may also lead to greater anisotropy in the magnetic properties (data not shown). The challenge to crystallize either a reduced catalyst without Cr or a Cr-containing catalyst suggests that both reduction and Cr incorporation likely affect polyoxometalate reassembling in solution (also suggested by the changes of environments, data not shown).

As such, the similarity of the spin systems yielding the anisotropic signals of <g>˜1.8 in FIG. 5, Spectra C and D, but absent in FIG. 5, Spectrum B, is even more interesting. The samples that yielded the spectra were reprecipitated from solutions following very different histories: H₃PW₁₂O₄₀ catalyst reduced by pyrolysis products from HDPE for FIG. 5, Spectrum C, and H₃PW₁₂O₄₀ catalyst reduced by H₂ first and used in three sequential runs with recovered spent catalyst for FIG. 5, Spectrum D. However, each of the two segments within 360-420 mT could be fitted with two uncoupled spin systems of similar principal components of anisotropic g tensors with major differences in weight (population) and broadening (data not shown). Alternative fitting attempts missed the substructures around each peak near 385 mT (data not shown). Correlating with the respective carbon number distribution of products corresponding to these samples, the <g> values of ˜1.8 evident in FIG. 5, Spectra C and D spectra but absent in FIG. 5, Spectrum B suggests the effect of electron delocalization on proton mobility, and consequently on the reactivity observed for HDPE cracking. This is also reflected by the various quantities of NH₃ adsorption in water and methanol despite similar acid density determined by reverse titration (data not shown).

When H₂ is used to reduce H₃PW₁₂O₄₀, its oxidation is likely accompanied by one oxygen extraction and two electron transfer. Thus, the interaction of these electron spins with the 3 d spins of Cr likely played the dominant role in forming the spin systems (possibly an unpaired electron in the highest occupied molecular orbitals (HOMO) of each heteropolytungstate cluster, (data not shown) associated with FIG. 5, Spectrum D. However, the mechanism for H₃PW₁₂O₄₀ reduction by HDPE is more complex, as the foregoing results suggest that the olefins from HDPE pyrolysis are likely the actual reductants. Studies of the reduction of the oxides of Cr(VI), Mo(VI) and W(VI) by olefins revealed formation of mixed organic molecules. They could possibly involve or form intermediates, such as radicals or singlet alkylidenes, which are drastically more stable than intermediates from H₂, and may serve as reducing agents to transfer odd numbers of electrons per heteropolytungstate unit. For example, through hydrogen transfer (i.e., a proton and an electron) across multiple units mediated by these intermediates, only a fraction of units lose oxygen atoms to achieve overall charge balance.

In summary, the EPR spectroscopy results support multiple effects of Cr, including modified atomic and electronic structures of the polyoxometalate catalysts. Reducibility and delocalized electron density can possibly modify the acidity of the materials (data not shown), which in turn affects the reactivity and selectivity during catalysis. The effects are likely applicable to supported catalysts in general. For example, the Cr from polymerization catalyst and 10²-10³ ppm pigments (such as Cr₂O₃ and PbCrO₄) could possibly anchor on oxides and lead to surface or subsurface changes of reducible supports used in other catalysts for polyolefin upcycling, while affecting the electronic structures around the active centers and the oxide supports. Hence, it is vital to consider such effects due to transition metal accumulation in catalysis for applications including but not limited to polyolefin upcycling. Additionally, in other applications that harness catalyst acidity, the reduction of polyoxometalates at high temperatures possibly results in increased coke formation and activity degradation.

### CONCLUSION

This Example successfully demonstrates facile cracking of molten HDPE by 12-tungstophosphoric acid and its acidic derivatives in an up-flow tubular reactor. In the 573-673 K reaction temperature range, a hydrocarbon crude (>90% C₃-C₃₀ paraffins yield) could be obtained. The carbon number distribution of the product can be shifted between heavier and lighter hydrocarbons by controlling the product reflux zone temperatures in the effluent of the vertical up-flow reactor. Higher reflux zone temperatures allowed heavier cracked products to reach the product condenser, while lower reflux zone temperatures allowed more of the products to undergo repeated cracking on the catalyst. The spent catalyst was recovered by selectively dissolving in water, filtering off the coke, and reprecipitating from the filtrate. Coke formation increased upon dehydrogenation of the substrate and its cracked products and could encapsulate the catalyst, impeding its recovery. Such dehydrogenation was promoted by polyoxometalates containing certain metal(s) at the highest oxidation state (such as +6 for W and Mo, +5 for V). Catalyst pre-reduction with either hydrogen or cracking products drastically mitigated such dehydrogenation to enable facile catalyst recovery and reuse, while also increasing the overall cracking activity.

This Example further demonstrates that trace Cr metal present in the HDPE sample accumulates in the cracking catalyst, likely affecting both atomic and electronic structures as well as the reducibility of the catalyst. With each recycle, the increased Cr content in the catalyst aided the formation of lighter paraffins. However, XRD results revealed that excess Cr reduced the stability of the cage-like structure of the catalysts pre-reduced in H₂. Intentionally doping 12-tungstophosporic acid with various amounts of chromium nitrate revealed the disintegration of a small fraction of the Keggin structure with ˜630 ppm Cr. In contrast, pre-reduction using olefins (also present in the pyrolysis products from polyolefins) not only increased the production of lighter paraffins but also preserved the cage-like structure of the catalysts up to a Cr mass content of ˜914 ppm. EPR spectroscopic studies of the various fresh and recovered catalysts revealed a correlation between enhanced cracking activity and unpaired delocalized electrons. The results clearly point to the need for controlling the accumulation of metal impurities (such as Cr) in the catalyst for maintaining a desired product distribution and proper disposal/recycle of the excess metal for the overall sustainability of plastics upcycling processes. The insights from this Example provide guidance for future investigations aimed at better understanding the role of Cr on catalyst structure, stability, reducibility, and activity, as well as strategies to remove Cr.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

Unless otherwise indicated, the term “type” as used herein refers to chemical formula such that a single type means the same chemical formula and different type means different chemical formula. Similarly, use of “more” as in “one or more” refers to use of different types of the relevant entity.

Throughout the present disclosure, terms such as “comprising” and the like may be replaced with terms such as “consisting” and the like.

What is claimed is:

1. A method for cracking a polyolefin, the method comprising:
(a) heating a blend comprising a polyolefin and an unsupported acidic polyoxometalate in a reaction zone of a reactor chamber, the reaction zone at a reaction temperature, while flowing a gas through the blend, to induce carbon-carbon bond cleavage in the polyolefin and form a processed blend comprising cracked hydrocarbons;
(b) passing at least a portion of the processed blend to a reflux zone of the reactor chamber, the reflux zone at a reflux temperature; and
(c) collecting the cracked hydrocarbons.

2. The method of claim 1, wherein the polyolefin comprises polyethylene, polypropylene, polyisobutylene, or a combination thereof.

3. The method of claim 1, wherein the polyolefin has a weight average molecular weight of at least 500 kDa.

4. The method of claim 1, wherein the polyolefin has a crystallinity of at least 70%.

5. The method of claim 1, wherein the polyolefin has a weight average molecular weight of at least 1000 kDa and a crystallinity of at least 70%.

6. The method of claim 1, wherein the polyolefin is provided by pieces of a consumer product.

7. The method of claim 1, wherein the unsupported acidic polyoxometalate comprises W, Mo, V, or combinations thereof.

8. The method of claim 1, wherein the unsupported acidic polyoxometalate has a formula HₓM₃₋ₓPM′_{y}W_{12-y}O₄₀, wherein M is an alkali metal, M′ is Mo or V, y is from 0 to 12, and x is from 0 to 3.

9. The method of claim 1, wherein the unsupported acidic polyoxometalate is H₃PW₁₂O₄₀.

10. The method of claim 1, wherein the reactor temperature is greater than the reflux temperature.

11. The method of claim 10, wherein the reactor temperature is in a range of from 500 K to 700 K and the reflux temperature is from 300 K to 475 K.

12. The method of claim 1, wherein the unsupported acidic polyoxometalate has been reduced prior to step (a).

13. The method of claim 12, further comprising reducing the unsupported acidic polyoxometalate by exposing the unsupported acidic polyoxometalate to a reductant while heating.

14. The method of claim 13, wherein the reductant is H₂, an olefin, or a combination thereof.

15. The method of claim 13, wherein the reductant is an olefin provided by products of polyolefin thermal pyrolysis.

16. The method of claim 1, wherein the unsupported acidic polyoxometalate is doped with a transition metal selected from Ti, Cr, Zr, Fe, and combinations thereof.

17. The method of claim 16, wherein the transition metal is present at an amount in a range of from 20 ppm to 1000 ppm.

18. The method of claim 1, further comprising recovering the unsupported acidic polyoxometalate after step (c) by exposing the unsupported acidic polyoxometalate to a protic solvent to form a recovery solution comprising dissolved unsupported acidic polyoxometalate; filtering the recovery solution to provide a filtrate; and precipitating the unsupported acidic polyoxometalate from the filtrate and using it to carry out steps (a)-(c) one or more additional times.

19. The method of claim 1, characterized by an overall product yield of at least 80% wherein at least 80% of the cracked hydrocarbons comprise olefins and paraffins.

20. The method of claim 1, wherein the polyolefin has a weight average molecular weight of at least 1000 kDa and a crystallinity of at least 70%; the polyolefin is provided by pieces of a consumer product; the unsupported acidic polyoxometalate has a formula HₓM₃₋ₓPM′_{y}W_{12-y}O₄₀, wherein M is an alkali metal, M′ is Mo or V, y is from 0 to 12, and x is from 0 to 3; and the reactor temperature is in a range of from 500 K to 700 K and the reflux temperature is from 300 K to 475 K.
